(19) United States
(12) Patent Application Publication
(10) Pub. No.: US 2004/0186513 A1
(43) Pub. Date: Sep. 23, 2004

(54) Medical instrument

(75) Inventors: Manfred Dworschak, Duerbheim (DE); Theodor Lutze, Balgheim (DE); Pedro Morales, Tuttlingen-Nendingen (DE); Dieter Weisshaupt, Immendingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/767,310

(22) Filed: Jan. 29, 2004

Related U.S. Application Data
(63) Continuation of application No. PCT/EP02/08495, filed on Jul. 31, 2002.

(30) Foreign Application Priority Data
Aug. 4, 2001 (DE) 101 38 394.0

(51) Int. Cl. A61B 17/28; A61B 17/44

(52) U.S. Cl. 606/208

(57) ABSTRACT

A medical instrument is provided which comprises a first part and a second part which are connected together by a connecting device. The second part abuts on the first part. A cover element which is connected to the first part is disposed on the second part in such a manner that relative movement of the second part away from the first part is blocked by the cover element.

The present disclosure relates to the subject matter disclosed in German application No. 101 38 394.0 of Aug. 4, 2001 and international application No. PCT/EPO2/08495 of Jul. 31, 2002, which are incorporated herein by reference in their entirety and for all purposes.

### BACKGROUND OF THE INVENTION

The invention relates to a medical instrument having a first part and a second part which are connected together by a connecting device.

Hereby for example, this may be a pair of forceps, particularly in the form of a clamp.

### SUMMARY OF THE INVENTION

In accordance with the invention, a medical instrument is provided in which the connection is manufactureable in a simple and economical manner.

This is achieved in that the second part abuts on the first part and in that there is provided a cover element (lid element) which is connected to the first part and which is disposed on the second part (put on the second part) in such a manner that a relative movement of the second part away from the first part is blocked by the cover element.

The arrangement of the second part on the first part blocks the movement of the second part towards the first part and the cover element blocks a movement in the opposite direction. A secure, and in particular, a substantially play-free connection between the two parts can thereby be achieved.

But then, on the other hand, a relative movement of the two parts in directions transverse to this direction of connection can still be produced by the provision of, in particular, appropriate guidings. Thus, for example, the facility for a linear displacement between the two parts could be provided or the two parts can be constructed such as to be rotatable relative to one another.

The corresponding connecting device can also be employed in a simple manner in a medical instrument which consists essentially of a synthetic material. For example, the cover element can be connected to the first part by gluing, welding, a snap-action closure or the like.

A corresponding instrument can also be produced in a simple manner since the two parts are firstly placed upon one another and the cover element then secures this position. The cover element can be put on after the two parts have been placed upon one another.

In particular, it is advantageous if a portion of the surface of the second part abuts on an associated portion of the surface of the first part in order thus to block a relative movement of the second part towards the first part.

Furthermore, it is advantageous if a portion of the surface of the cover element abuts on an associated portion of the surface of the second part in order thus to block a relative movement of the second part away from the first part.

In an advantageous variant of an embodiment, one part is provided with a spigot (shaft end) and the other part with a spigot recess for accommodating the spigot. In particular, a guiding for the second part relative to the first part by means of the connecting device can be formed in this manner.

In a first exemplary embodiment, the cover element is placed on (put on) the spigot. Then, on the one hand, the spigot serves for the guidance of the two parts relative to one another, for example, for the purpose of forming a pivotal guide means, whilst the cover element can then be connected to the spigot in order to fix the connection between the two parts on the other.

Basically, the cover element can be connected to the first part and in particular, to its spigot, in different ways, for example, by adhesion, a screw connection, welding or by means of thermal connecting techniques. A medical instrument in accordance with the invention can be assembled in a simple manner if the cover element is adapted to be seated on the spigot in the manner of a snap-action closure. When producing the medical instrument, the second part is then laid upon the first part such that a spigot recess in the second part is aligned with the spigot on the first part. The subsequent application of the cover element secures this position so that movement of the second part away from the first part in the longitudinal direction of the spigot is blocked. In particular, a connection of this type can then be formed such that it is substantially free from play.

Provision may be made for the spigot to be provided with one or more longitudinal slots in order to form a seating for the cover element in the manner of a snap-action closure. The effect is thereby obtained that corresponding parts of the spigot can spring back as a result of the force that is applied when putting on the cover and they will then engage in a corresponding recess in the cover. The cover itself can then only be released from the spigot by the application of force.

It is expedient hereby, if the cover element has a larger transverse dimension than the spigot at least in the region of a contact surface with the second part. This thus provides a corresponding contact surface with the aid of which the second part is adapted to be blocked from moving away from the first part.

Hereby, in correspondence with the contact surface of the cover element, a contact surface for the cover element is arranged on the second part, and in particular in the spigot recess in order to accordingly provide the necessary contact surface for blocking the relative movement.

In a variant of the embodiment, the spigot is arranged on the first part and the spigot recess is arranged in the second part. A relative rotatability between the two parts can be realized in this manner.

In a second exemplary embodiment, the first part comprises a seating in which the second part is adapted, at least in part, to be placed on the first part.

The cover element then blocks withdrawal of the second part from the seating, i.e. the second part lies on a facing surface of the cover element.

Provision may be made for the second part to be moveable relative to the first part in a seating. Hereby, the seating can be formed in the second part itself or in the first part. In particular thereby, a means for guiding the second part on the first part is provided, this means being, for example, a linear guide means or a pivotal guide means.

Provision may be made for one part to comprise at least one spigot and for the other part to comprise at least one seating for the purposes of guiding or holding the other part on said one part. For example, if a spigot is held in a corresponding spigot seating in rotatable manner, then a pivotal guide means is thereby formed. If a spigot is mounted in a corresponding seating which is constructed in the manner of a longitudinal slot, then a displacement guide means can thereby be formed. Should spaced spigots have entered into corresponding recesses in the other part (or, if one part comprises a spigot and a recess and the other part comprises a corresponding recess and a corresponding spigot), then the facility for movement in the seating is thereby blocked due to the connections formed by the spigot and the spigot recess, and the two parts are held together so as to be relatively immovable.

In particular, the two parts are arranged to be moveable relative to one another via the connecting device. Accordingly for example, a pivotal guide means is formed by means of a spigot in the form of a shaft.

The two parts are then connected together in rotatable manner. A pair of forceps and then, as a special form thereof, a clamp for example, can be constructed in this manner by means of the connecting device in accordance with the invention.

However, a pair of tweezers could also be formed by means of a fixed connection between a first part and a second part.

It is quite particularly advantageous, if the first part and/or the second part are made of a synthetic material and, in particular, if they are formed in one piece in each case. The corresponding connecting device can then be produced in a simple manner.

In principle, provision may be made for the cover element to be a separate part which is connectable to the first part by, for example, gluing, welding or by means of a latching connection. However, provision could also be made for the cover element to be seated on the first part in one piece manner and, in particular, is thereby formed integrally therewith during the production of the first part. Thus, a corresponding medical instrument can be made from just tow separate parts, namely, the first part and the second part, and, moreover, without the need for still further additional parts.

In particular, the cover element is then seated on the first part in pivotal manner for the purposes of being placed on the second part. Then, for example, the cover element can be moved away from a seating for the second part to such an extent that it can be inserted flat into the seating or the like. If the cover element is then pivoted onto the second part, then it can be laid on the second part in order, to block the second part from moving away.

The cover element can be constructed such that it is pivotal on the first part in a simple manner if it is seated on the first part by means of a film hinge.

The cover element arranged in one piece manner on the first part can be fixed to the first part in order to produce a blocking connection for the second part, i.e. it can, in particular, be fixed to the first part in a connecting region of the cover element whereat the cover element is not connected in one piece manner to the first part. The cover element is then fixable to the first part in such a manner that a movement away therefrom is blocked so as, in turn, to block a reverse movement of the second part by virtue of it resting on the second part. If the cover element is seated on the first part in pivotal manner, then this form of fixing via the connecting region thereby connects the free end of the cover element to the first part.

The cover element can be fixed to the first part in a simple and easily produced manner if a latching connection is provided. This can be produced in a rapid manner, without the need for a complex treatment such as welding for example, or without the necessity to provide adhesives.

It is also expedient if the cover element comprises a coupling element and if the first part comprises a corresponding coupling element which are matched and adapted to one anther in such a manner that, in the case of a fixed cover element, a transverse movement of the cover element relative to the first part taken with reference to the direction in which it is applied is blocked by virtue of the co-operation between the coupling elements. Transverse forces on the first part in a direction transverse to the direction of application can be accommodated by virtue of these corresponding coupling elements. Consequently, the latching connection does not have to accommodate the transverse forces, at least not to their full extent, and, by appropriate construction of the coupling elements, the predominant part of the transverse forces can be intercepted thereby. The latching connection can thereby be optimised in regard to its function as a fixing means between the cover element and the first part. In turn, the assembly of a medical instrument in accordance with the invention can thereby be facilitated since the fixing connection between the two parts is producible in a simple manner.

Thus, in an expedient embodiment, the coupling elements co-operate in the manner of a seating and a corresponding element which enters the seating in order to enable the transverse forces to be accommodated and thus to enable them to be diverted from the latching connection to a considerable degree. Such a first part can also be produced in a simple manner in one piece with the corresponding cover element.

In conjunction with the drawing, the following description of preferred embodiments serves to provide a more detailed explanation of the invention.

### BRIEF DESCRIPTION OF THE DRAWINGS

FIG. 1 shows a plan view of a pair of forceps which is provided with a connecting device in accordance with the invention;

FIG. 2 a sectional view through the connecting device depicted in FIG. 1;

FIG. 3 a partial view of a medical instrument which is provided with a second exemplary embodiment of a connecting device in accordance with the invention;

FIG. 4 a sectional view along the line A-A depicted in FIG. 3;

FIG. 5 a partial, sectional view along the line B-B depicted in FIG. 3 in the form of an exploded view;

FIG. 6 a partial view of an endoscopic instrument, wherein a first part and a second part are connected by means of a connecting device in accordance with the invention;

FIG. 7 a partial side view of a medical instrument which is provided with a third exemplary embodiment of a connecting device in accordance with the invention in the case where the cover element is not fixed;

FIG. 8 the same view as FIG. 7 in the case of a fixed cover element;

FIG. 9 a sectional view along the line 9-9 depicted in FIG. 8 and

FIG. 10 a perspective partial view of the connecting device in accordance with the third exemplary embodiment.

### DETAILED DESCRIPTION OF THE INVENTION

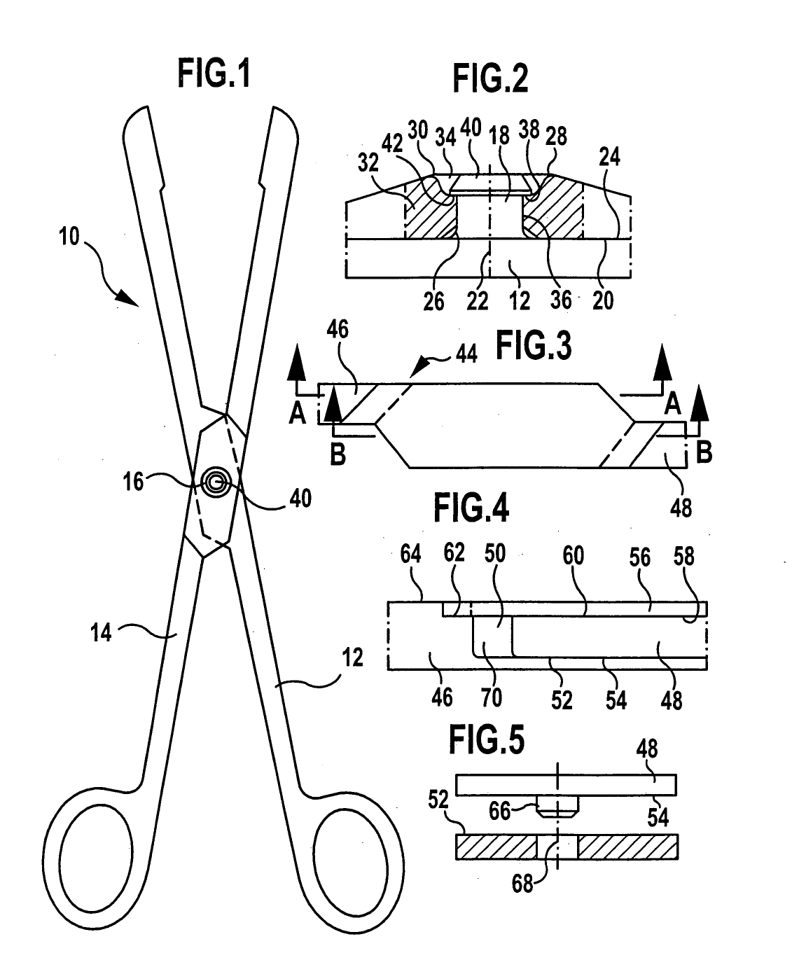

An exemplary embodiment of a medical instrument is formed by a clamp which bears the general reference 10 in FIG. 1. This comprises a first part 12 in the form of a first arm and a second part 14 in the form of a second arm. These arms are connected together by a connecting device 16, whereby this connecting device 16 comprises a joint so that the first part 12 and the second part 14 are pivotal relative to one another.

Here, a spigot 18 (shaft end), which projects above an upper face 20 of the first part 12 facing the second part 14, is arranged on the first part 12 (FIG. 2). This spigot 18 may be formed in one piece with the first part 12; for example, the first part 12 (and also the second part 14) is made from a synthetic material and in particular hereby, each part 12, 14 is formed in one piece and, during the production of the first part 12 by means of an injection moulding process for example, the spigot 18 is formed therewith by appropriate shaping of the mould.

However, it could also be mounted on the first part 12 at a later time point in that, for example, the spigot 18 is provided with a thread and is screwed into the first part 12.

The spigot 18 extends thereby in a longitudinal direction 22 from the upper face 20 of the first part 12.

The second part 14 has a lower face 24 which is matched to the upper face 20 of the first part 12 so that the lower face 24 of the second part 14 can lie on the upper face 20 of the first part 12. The relative movement of the second part 14 towards the first part 12 is blocked by such an arrangement.

In dependence on the production method, the spigot 18 may comprise a widened flank 26 at the point of transition to the upper face 20 of the first part 12 so that the cross section of the spigot 18 in the vicinity of the flank 26 is greater than it is beyond this flank 26.

In particular, the spigot 18 is rotationally symmetrical about the axis 22.

The second part 14 has a spigot recess 28 for accommodating the spigot 18. This extends, in particular, between the lower face 24 of the second part 14 and an upper face 30 thereof. Hereby, the thickness of the second part 14 in a portion 32 of the second part 14 surrounding the spigot recess 28 is greater than it is outside the portion 32.

The spigot recess 28 is matched to the dimensions of the spigot 18 so that the latter can be accommodated by the spigot recess 28. In particular, the spigot recess 28 is widened in the direction towards the lower face 24 so that the flanks 26 can also be accommodated by the spigot recess 28.

At the upper face 20, the spigot recess 28 has a widened portion 34 which is of greater cross-section than the spigot 18 and that portion 36 of the spigot recess 28 within which the spigot 18 is guided with the walls thereof substantially parallel to the direction 22. There is thus formed on the spigot recess 28 a contact surface 38 which is, in particular, of annular shape.

A cover element 40 (lid element), whose surface 42 facing the upper face 20 of the first part 12 lies on the contact surface 38, is placed on the spigot 18. Hereby, in the case of an annular contact surface 38, the corresponding part of the surface 42, which forms a contact surface for the cover element 40, is likewise annular.

In particular, the cover element 40 is formed in such a manner that it is disposed within the region 34 of the spigot recess 28 so that it does not project above the upper face 30 of the second part 14.

The cover element 40 may be stuck to the spigot 18, welded thereto, screwed thereto or be connected thereto by means of a thermal bond.

In the case of a variant of an embodiment, the cover element 40 is clipped onto the spigot 18 in the manner of a snap-action closure.

Provision may then be made for the spigot 18 to be provided with appropriate slots so that the cover element 40 is insertible into a corresponding recess in the spigot 18 and is then held therein by snap-action in the manner of a snap-action closure.

The connection of the second part 14 to the first part 12 is produced by means of the connecting device 16 in such a manner that the second part 14 is put on the first part 12 with the spigot recess 28 therein aligned with the spigot 18. The movement of the second part 14 in the direction of the first part 12 is thereby blocked. The cover element 40 is subsequently placed on the spigot 18 and fixed in position there, for example, by means of a snap-action closure. In turn, the contact surface of the surface 42 of the cover element 40 thereby presses on the contact surface 38 within the spigot recess 28 in the second part 14. Movement of the second part 14 away from the first part 12 is thereby blocked and secure fixing of the second part 14 to the first part 12 is achieved.

In the case of the exemplary embodiment shown in FIGS. 1 and 2, the connecting device 16 is in the form of a joint, i.e. the second part 14 can be pivoted relative to the first part 12 by means of the spigot 18 in the spigot recess 28 which is in the form of a rotary bearing for the rotary In a second exemplary embodiment of a connecting device in accordance with the invention which bears the general reference 44 in FIG. 3, a first part 46 and a second part 48 are connected to one another. Here, a seating 50 for the second part is formed in the first part 46 (FIG. 4). This seating 50 has a contact surface 52 for the second part 48 so that a lower face 54 of the second part 48 is adapted to be laid upon the contact surface 52. The movement of the second part 14 in the direction of the first part 12 is thereby blocked. A cover element 56 (lid element), whose surface 58 lies on an upper face 60 of the second part 48, is seated on the first part 12. Movement of the second part 14 away from the first part 12 is thereby blocked and the connecting device provides for the connection of the two parts 12 and 14.

The cover element 56 itself is fixed to the first part 12, for example, by adhesion, a snap-action closure, welding or a positive connection such as a screw-type connection or by means of a thermal fixing process.

In the case of the exemplary embodiment shown in FIG. 4, the first part 46 has a recess 62 whose height corresponds to the height of the cover element 56. The cover element 56 can then be inserted into this recess and an appropriate counterpart (not shown in the drawing) and it can thereby lie on the second part 48 whilst held on the first part 46 so that a surface 64 of the first part 12 together with the cover element 56 that is fixed thereto will have substantially no projections, and in particular, at the point of transition to the cover element 56.

The seating 50 may be formed in such a manner that the second part 48 also lies on the boundary walls of the seating 50 in transverse directions (perpendicular to the plane of the drawing in FIG. 4). The second part 48 is thereby held firmly on the first part 46.

In particular, provision may however be made, as shown in exemplarily manner in FIG. 5, for the second part 48 to be arranged in the seating 50 such that it is moveable relative to the first part 46. For example, a displacement guide means can be provided, the second part 48 being guided therein in displaceable manner relative to the first part 46, for example in parallel with a longitudinal direction of the first part 46 (not shown in the drawing).

In particular, as is shown in FIG. 5, provision is made for the second part 48 to be rotatable relative to the first part 46. To this end, the second part 48 is provided with a spigot 66 which faces the first part 46 and enters a corresponding recess 68 in the first part 46. This spigot 66 then represents a rotary shaft which is guided for pivotal motion in the recess 68. For the purposes of executing the rotary movement, an appropriate space 70 must be made available in the seating 50 (compare FIG. 4) so that the second part 48, and in particular an end portion thereof, will not strike against the boundary walls of the seating 50 before the set maximum pivotal angle is reached.

Alternatively, a spigot serving as a rotary shaft can be arranged on first part 46 in the direction of the second part 48, whereby the second part 48 then comprises a corresponding recess.

Furthermore, a plurality of spigots and recesses can be provided, also in alternating manner, i.e. the first part 46 is provided with recesses and spigots and, in correspondence therewith, the second part 48 is provided with corresponding spigots and recesses. If a plurality thereof is provided, then the second part 48 is held firmly on the first part 46 by means of the spigots and recesses in this manner, whereby movement of the second part 48 away from the first part 46 is secured by the cover element 56.

The connecting device 44 in accordance with the invention can be produced on a medical instrument in a simple manner by manufacturing the first part 46 and the second part 48 from a synthetic material.

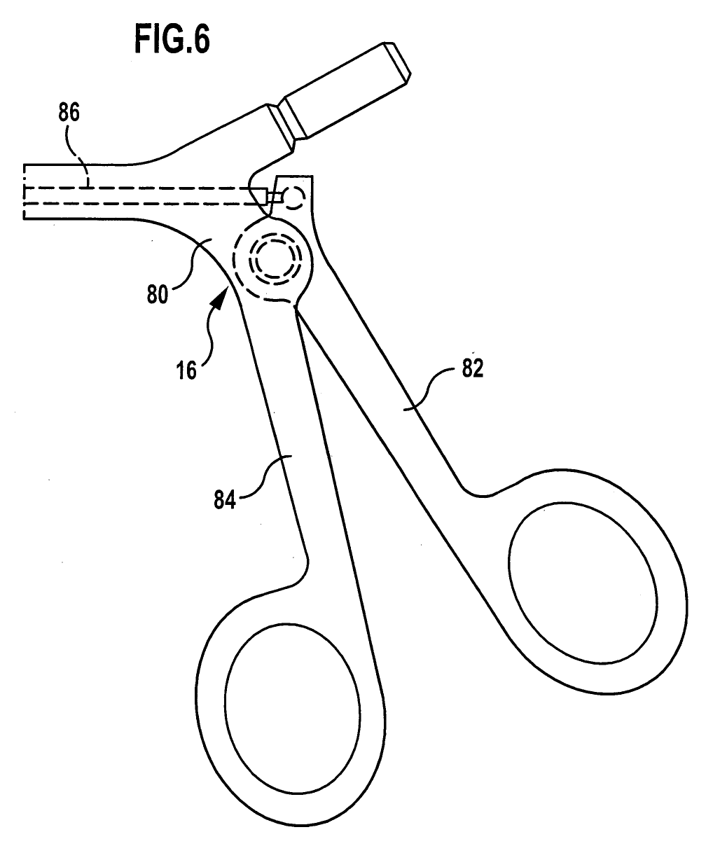

A further exemplary embodiment of a medical instrument wherein a first part 80 and a second part 82 are connected together by means of a connecting device 16 or 44 in accordance with the invention is shown in FIG. 6. Here, this is an endoscopic instrument wherein a handle element 84 is arranged on the first part 80 and the second part 82 represents a further handle element which is pivotal relative to the handle element 84 via the connecting device 16 or 44. An actuating element 86 can thereby be displaced in the longitudinal direction thereof by virtue of the pivotal movement of the handle element 82.

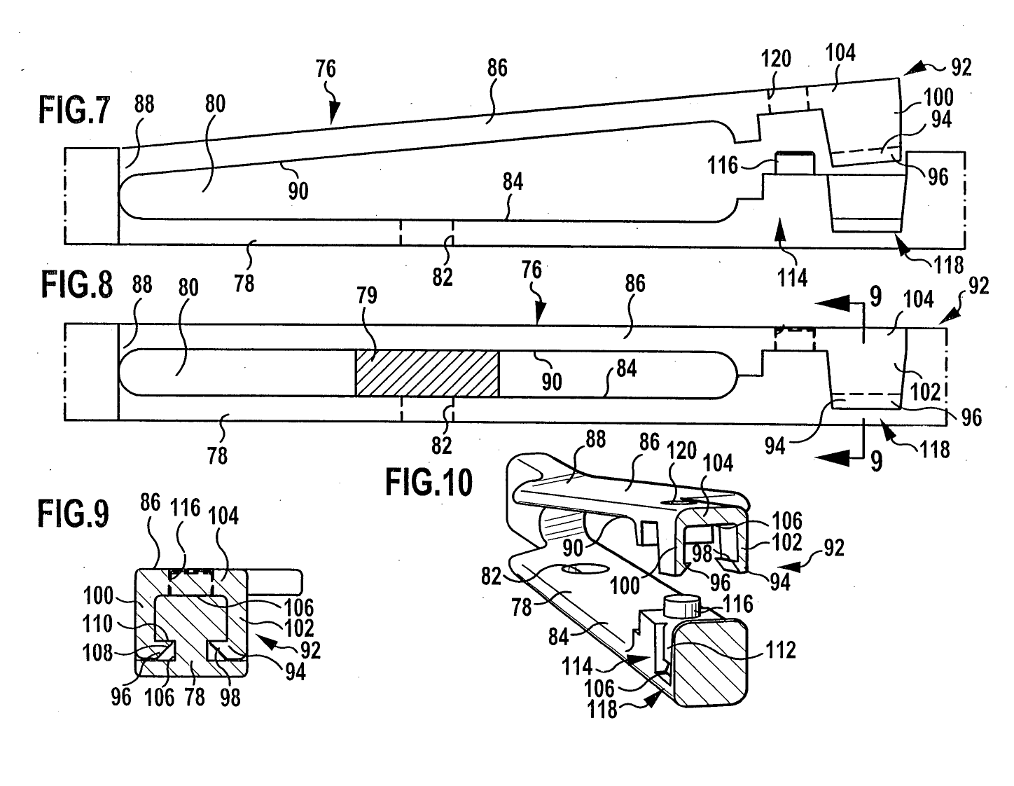

In a third exemplary embodiment of a connecting device in accordance with the invention which is shown in FIGS. 7 to 10 and bears the general reference 76 in FIGS. 7 and 8, a seating 80 for a second part 79 that is shown in FIG. 8 is formed on a first part 78. The second part 79 is arranged within this seating 80 in longitudinally displaceable or pivotal manner for example, and an appropriate displacement guide means or pivotal guide means is provided. In the case of the exemplary embodiment shown, the first part 78 facing the seating 80 comprises a spigot seating 82 which serves as a pivotal bearing for accommodating a spigot in the form of a stub shaft that is formed on the second part 79.

The seating 80 is bounded at one side by a substantially flat surface 84 upon which the second part 79 is adapted to be laid in order to provide a surface for blocking relative movement between the second part 79 and the first part 78 in the direction of the surface 84

A cover element 86, which was formed integrally during the production of the first part 78, is seated on the first part 78 and is connected thereto in one piece manner. For example, this cover element 86 is arranged to be pivotal on the first part 78 by means of a film hinge 88.

The cover element 86 comprises a substantially flat surface 90 which faces the surface 84 and bounds the seating 80 and which serves as a surface for blocking movement of the second part 79 away from the first part 78 in the direction of the surface 90. The cover element 86 is laid on the second part 79 via the surface 90 or at least a portion of this surface (FIG. 8).

The cover element 86 is adapted to be fixed to the first part of a latching connection 92. To this end, the cover element 86 is provided with a latching nose device 94 which is arranged at an end of the cover element 86 that is remote from the region of the cover element 86 connected to the first part 78 by means of the one piece connection thereof, for example, the film hinge 88.

In the case of the exemplary embodiment shown, the latching nose device 94 comprises laterally opposite latching noses 96, 98 which are formed on laterally opposite, spaced walls 100, 102. Hereby, these walls 100, 102 project beyond the surface 90 in the direction of the surface 84. The walls 100, 102 are connected by a connecting part 104 of the cover element 86 which provides a contact surface 106 between the two walls 100 and 102.

The latching noses 96, 98 project towards one another from the inner surfaces of their respective walls 100, 102.

A correspondingly shaped recess 106 is formed in the first part 78 for the latching nose device 94 in order to accommodate the latching noses 96, 98, namely, in the manner of an undercut so that, when the latching noses have entered the corresponding recesses, a movement of the cover element 86 is blocked in a direction opposite to the direction in which it was applied (for example, in the opposite direction to a pivotal direction of the cover element 86 towards the surface 84). Particularly to this end, the respective latching noses 96, 98 are formed with an inclined surface 108 that is arranged at an angle to the surface 90 and with a surface 110 that is parallel to the surface 90, whereby the inclined surface 108 is at a vertical angle to the parallel surface 110. The respective latching noses 96, 98 can thereby be inserted into their associated recess 106 by forming the walls 100, 102 of the cover element 86 in an appropriately flexible manner, whereby however, after they have engaged in this recess 106, their emergence therefrom by pivoting away in a direction opposite to the direction in which they were applied is blocked

In order to prevent the cross-section of the first part 78 being enlarged in the vicinity of the latching connection 92 in the case of a fixed cover element 86, parallel recesses 112 are provided for accommodating the walls 100 and 102, these recesses then being appropriately matched to the thickness of the walls 100, 102 so that, in the case of a fixed cover element 86, the exterior surfaces of the walls 100, 102 will be substantially flush with the corresponding further exterior surfaces of the first part 78 which are adjacent to the seating 80.

The first part 78 is provided with a coupling element in the form of a spigot 116 in a coupling region 114 following on the seating 80. Hereby, this coupling region 114 is located between the seating 80 and a latching region 118 which is provided with the recesses 106 and 112 for accommodating the latching noses 96, 98 and the walls 100, 102.

A seating 120 for the spigot 116 is formed in the cover element 86 as a coupling element corresponding to the spigot 116. If the cover element 86 is fixed to the first part 78 by means of the latching connection 92 in non-rotational manner, then the spigot 116 has entered the seating 120. Hereby, the spigot 116 extends in a direction which is transverse and substantially perpendicular to the surface 84. In the case of the exemplary embodiment shown, the spigot 116 itself is already at a distance from the surface 84, i.e. it has been displaced in height with respect thereto.

If the spigot 116 has entered the seating 120, then the movement of the cover element 86 relative to the first part 78 is blocked in a direction transverse to the direction in which the cover element 86 was applied to the second part 79. The latching connection 92 need then only accommodate substantially smaller transverse forces so that, in essence, the latching connection 92 is adapted to be optimised such as to achieve the effect that the cover element 86 is fixed to the first part 78 in latching manner, i.e. a reverse pivotal movement of the cover element 86 in a direction opposite to the direction in which the first part 78 was applied to the second part 79 is blocked. Since the transverse forces are substantially accommodatable by the coupling of the spigot 116 and the spigot seating 120, then, for example, no unusual precautions need to be taken in order to provide for lateral stability of the walls 100, 102 with respect to the connecting part 104. In turn, the walls 100, 102 can thereby be resilient and/or be arranged resiliently on the connecting part 104 in such a manner that the latching connection 92 is attainable in a simple manner by the entry of the latching noses 96, 98 into the associated recesses 112.

In the case of a non-fixed cover element 86 (FIG. 7), i.e. in the case of a cover element 86 which is pivoted in a direction opposite to the direction in which it was applied to such an extent that the second part 79 can be inserted into the seating 80 and accordingly, by virtue of a spigot entering the spigot seating 82 for example, a corresponding medical instrument is then produced just by moving the second part 79 into the seating 80. The cover element 86 is then moved towards the second part 79 in the direction of application, i.e. it is pivoted towards it for example, and the latching connection 92 and the coupling between the spigot 116 and the seating 120 is thereby produced (FIG. 8).

Simultaneously thereby, the cover element 86 is placed on the second part 79 via the surface 90 in order to block the movement of the second part 79 out of the seating 80 in the direction of the cover element 86 in this manner.

1. A medical instrument comprising:
a first part and a second part which are connected together by a connecting device;
wherein the second part abuts on the first part; and
a cover element which is connected to the first part and is disposed on the second part in such a manner that relative movement of the second part away from the first part is blocked by the cover element.

2. A medical instrument in accordance with claim 1, wherein a portion of the surface of the second part abuts on an associated portion of the surface of the first part.

3. A medical instrument in accordance with claim 1, wherein a portion of the surface of the cover element abuts on an associated portion of the surface of the second part.

4. A medical instrument in accordance with claim 1, wherein one part out of the first part and second part is provided with a spigot and the other part is provided with a spigot recess for accommodating the spigot.

5. A medical instrument in accordance with claim 4, wherein the cover element is positioned on the spigot.

6. A medical instrument in accordance with claim 4, wherein the cover element is adapted to be placed on the spigot in the manner of a snap-action closure.

7. A medical instrument in accordance with claim 4, wherein the spigot is provided with one or more longitudinal slots in order to form a seating for the cover element in the manner of a snap-action closure.

8. A medical instrument in accordance with claim 4, wherein the cover element has a larger transverse dimension than the spigot at least in the region of a contact surface on the second part.

9. A medical instrument in accordance with claim 8, wherein a contact surface for the cover element on the second part is arranged at the spigot recess.

10. A medical instrument in accordance with claim 4, wherein the spigot is arranged on the first part and the spigot recess is arranged in the second part.

11. A medical instrument in accordance with claim 1, wherein the first part comprises a seating in which the second part is adapted, at least in part, to be placed on the first part.

12. A medical instrument in accordance with claim 1, wherein the cover element blocks withdrawal of the second part from the seating.

13. A medical instrument in accordance with claim 1, wherein the second part is moveable relative to the first part in a seating.

14. A medical instrument in accordance with claim 13, having a guiding for the second part on the first part.

15. A medical instrument in accordance with claim 1, wherein one part out of the first part and second part comprises at least one spigot and the other part comprises at least one spigot seating for guiding or holding the other part on said one part.

16. A medical instrument in accordance with claim 1, wherein the two parts are moveable relative to one another via the connecting device.

17. A medical instrument in accordance with claim 16, wherein the two parts are connected together in rotatable manner.

18. A medical instrument in accordance with claim 1, wherein at least one of the first part and the second part are made substantially from a synthetic material.

19. A medical instrument in accordance with claim 1, wherein the cover element is seated on the first part in one piece manner.

20. A medical instrument in accordance with claim 1, wherein the cover element is seated on the first part in pivotal manner for the purposes of disposing it on the second part.

21. A medical instrument in accordance with claim 1, wherein the cover element is seated on the first part by means of a film hinge.

22. A medical instrument in accordance with claim 1, wherein the cover element is fixable to the first part.

23. A medical instrument in accordance with claim 22, wherein the cover element is fixable to the first part in such a manner as to block it from moving away therefrom.

24. A medical instrument in accordance with claim 22, wherein the cover element is fixable to the first part by means of a latching connection.

25. A medical instrument in accordance with claim 1, wherein the cover element comprises a coupling element and the first part comprises a corresponding coupling element which are matched to one another in such a manner that, in the case of a fixed cover element, a transverse movement of the cover element relative to the first part taken with reference to the direction in which it was applied is blocked by the co-operation between the coupling elements.

26. A medical instrument in accordance with claim 25, wherein the coupling elements co-operate in the form of a seating and a corresponding element which enters into the seating.

27. A medical instrument in accordance with claim 1, wherein, for the purposes of applying the cover element to the second part prior to being fixed to the first part, the cover element is moveable relative to the first part.

28. A medical instrument in accordance with claim 27, wherein the cover element is a separate part.

29. A medical instrument in accordance with claim 27, wherein the cover element is a part which is partially connected to the first part and is adapted to be fully fixed to the first part after being applied to the second part.
